(19) United States
(12) Patent Application Publication
(10) Pub. No.: US 2026/0162827 A1
(43) Pub. Date: Jun. 11, 2026

(54) COMPUTER-AIDED SYSTEM FOR DIAGNOSIS OF RENAL REJECTION

(71) Applicant: University of Louisville Research Foundation, Inc., Louisville, KY (US)

(72) Inventors: Ayman S. El-Baz, Louisville, KY (US); Ibrahim S. Abdelhalim, Louisville, KY (US); Sohail G. Contractor, Prospect, KY (US)

(21) Appl. No.: 19/414,950

(22) Filed: Dec. 10, 2025

Related U.S. Application Data
(60) Provisional application No. 63/730,686, filed on Dec. 11, 2024.

(51) Int. Cl. G16H 50/20; G06T 7/00; G16H 30/20; G16H 30/40

(52) U.S. Cl. CPC G16H 50/20; G06T 7/0016; G16H 30/20; G16H 30/40; G06T 2207/10088; G06T 2207/20081; G06T 2207/30084

(57) ABSTRACT

A non-invasive computer-aided system for detection of renal abnormalities indicative of graft rejection includes segmenting a medical image of a subject transplanted kidney, determining the apparent diffusion coefficient for the segmented kidney, creating 3D iso-surfaces based on the apparent diffusion coefficient, determining the cumulative distribution function of the apparent diffusion coefficient for each iso-surface, measuring the correlation between each cumulative distribution function, and, using a transformer-based machine learning model, classifying the subject kidney as normal or rejected based at least in part on the correlations.

### CROSS-REFERENCE TO RELATED APPLICATION

This application claims the benefit of priority to U.S. provisional patent application Ser. No. 63/730,686, filed Dec. 11, 2024, for COMPUTER-AIDED SYSTEM FOR DIAGNOSIS OF RENAL REJECTION, incorporated herein by reference.

### FIELD OF THE INVENTION

A non-invasive computer-aided system for detection of renal abnormalities indicative of graft rejection includes segmenting a medical image of a subject transplanted kidney, determining the apparent diffusion coefficient (ADC) for the segmented kidney, creating 3D iso-surfaces based on the ADC, determining the cumulative distribution function (CDF) of the ADC for each iso-surface, measuring the correlation between each CDF, and, using a transformer-based machine learning model, classifying the subject kidney as normal or rejected based at least in part on the correlations.

### BACKGROUND OF THE INVENTION

Renal transplantation, a primary procedure in organ transplantation, is the preferred treatment for end-stage renal failure. Despite progress, post-transplant complications and increased rates of renal insufficiency pose challenges to its effectiveness. Therefore, timely and accurate diagnosis and treatment are essential for the survival of transplanted kidneys, also referred to as grafts. Emerging imaging techniques like Magnetic Resonance Imaging (MRI) offer noninvasive assessment of transplanted kidneys. The ADC from Diffusion-Weighted Imaging (DWI) is a reliable biomarker for renal allograft function. High creatine clearance is indicative of kidney function as it reflects the kidney's efficiency in removing creatine from the blood. Consequently low blood creatine levels (i.e., high creatine clearance) correlate with a well-functioning kidney. Notably, patients with higher creatinine clearance typically have higher ADC values. It has been suggested that the use of Machine Learning (ML) with high-dimensional radiomics features of MRI can provide promising performance advantages, including improved diagnostic, prognostic, and predictive accuracy, which may lead to a rapid rise in the potential use of ML in renal imaging. Moreover, Deep Learning (DL) techniques have garnered attention in this field. However, existing work on this subject predominantly focuses on complete MRI scans and overlooks the importance of correlations between the iso-surfaces of the kidney. Furthermore, significant challenges exist, including ensuring consistency across Diffusion Weighted Magnetic Resonance Imaging (DW-MRI) scanning protocols at various magnetic field strength levels (e.g., within the range of 1 Tesla (T) and 3T) and addressing the shape deformation that may occur in the transplanted kidney, leading to alterations in MRI diffusion signals. Additionally, transformers, a type of DL architecture, hold promise for delivering more nuanced outcomes and have not been extensively utilized in this context. Moreover, there is a need for an expanded examination of different classifiers and their respective trade-offs. Also, renal transplant care is complex. Achieving a complete and accurate diagnosis during patient follow-up is an equally daunting task, fraught with challenges. The dynamic nature of graft health necessitates not just a one-time assessment but a series of exhaustive examinations using diverse MRI scanning protocols.

### SUMMARY

The present invention addresses these needs by introducing a transformer-based system for classifying acute renal transplant rejection using DW-MRI data obtained through various scanning protocols (such as, for example, 1.5T and 3T). The system streamlines the challenging process of examining diverse MRI scanning protocols, thereby enhancing the accuracy of follow-up diagnoses. The system can be broadly summarized as involving several steps. First, a preprocessing step includes segmentation of kidney DW-MRI image data and registration with a reference image. Next, ADC maps of the segmented kidney are generated. Then, iso-surfaces are generated from these maps. Next, the CDF is determined for each iso-surface. The Spearman correlation algorithm is then applied to these CDFs corresponding to iso-surfaces. Finally, a transformer-based machine learning model is employed to distinguish between normal and acutely rejected transplants based at least in part on the determined correlations. Greater variation in correlations is associated with abnormalities such as inflammation, fibrosis and ischemia in transplanted kidneys while lower variation in correlations is associated with normal kidney function. The transformer-based machine learning model learns these distinctions from nuanced, multivariate correlation patterns across various regions of the kidney rather than via a strict quantitative threshold, allowing for a more flexible classification system.

In one embodiment, the present invention is a novel transformer-based system for classifying acute renal transplant rejection using DW-MRI data obtained through various scanning protocols, which is a new approach that accounts for variations arising from differences in MRI magnetic field strength as well as other protocol-related factors such as differing imaging sequences and scanner models. These variations can impact the raw diffusion signals captured in DW-MRI, potentially leading to inconsistent ADC values and image characteristics across scans. The disclosed transformer-based system mitigates these effects by focusing on correlations, specifically, Spearman correlations of CDFs from ADC maps across kidney regions. This correlation-based approach improved the standardization of data interpretation by emphasizing relative patterns in kidney tissue properties rather than absolute values that may fluctuate due to scanner variations.

In another embodiment, the present invention is a Transformer-based Correlations to Classes Converter (T3C) model utilizing the correlations of the CDFs to distinguish between normal and acutely rejected transplants, highlighting the effectiveness and versatility of the T3C model in this context.

In a further embodiment, the present invention is a computer-aided method for diagnosis of renal rejection including receiving a plurality of medical images of a subject transplanted kidney; transforming each of the plurality of medical images into a corresponding apparent diffusion coefficient (ADC) map; identifying, for each ADC map, iso-surfaces in the ADC map; identifying correlations between iso-surfaces; classifying, using a machine-learning classifier, the subject kidney as normal or acutely rejected based at least in part on the correlations. In some embodiments, the plurality of medical images of the subject transplanted kidney are a plurality of DW-MRI scans of the subject kidney. In further embodiments, the machine-learning classifier is trained on a data set of DW-MRI scans of normal kidneys and acutely rejected kidneys obtained at a plurality of different magnetic field strengths. In certain embodiments, the method includes, prior to said transforming, selecting one of the plurality of medical images to be a reference image and aligning the remaining images in the plurality of medical images to the reference image. In some embodiments, identifying correlations between iso-surfaces comprises identifying correlations between cumulative distribution functions of each iso-surface. In further embodiments, the method includes determining the significance of each correlation and discarding correlations of lesser significance prior to the machine-learning classifier receiving the correlations as input. In certain embodiments, the method includes, subsequent to identifying correlations between iso-surfaces and prior to said classifying, defining an input series of tokens, each token comprising learnable position embeddings and correlation embeddings derived from the identified correlations between iso-surfaces. In some embodiments, the machine learning classifier includes a transformer encoder and a classifier. In further embodiments, the encoder maps the input series of tokens to a contextualized encoding sequence, and wherein the classifier classifies the subject kidney as normal or acutely rejected based on the contextualized encoding sequence. In certain embodiments, the transformer encoder includes a plurality of layers, each later including a multi-headed self-attention block followed by a point-wise Multi-Layer Perceptron block, with layer normalization (LN) applied before and residual connections added after each block.

In further embodiments, the present invention is a computer-aided system for diagnosis of renal rejection, the system including at least one non-transitory computer readable storage medium having computer program instructions stored thereon; and at least one processor configured to execute the computer program instructions causing the processor to perform the previously recited operations.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 depicts a schematic overview of the computer-aided system for diagnosis of renal rejection.

FIG. 2 is a series of cross-sections of 3D images of kidneys through the registration process, including (a) the reference image, (b) the moving image with respect to the reference image, (c) the moving image with respect to the reference image following a 12-degree of freedom affine-based registration, and (d) the moving image with respect to the reference image following a non-rigid 3D B-spline transformation.

FIG. 3 is a schematic illustration of the process flow from acquiring the iso-surfaces to calculating the corresponding correlations in (a) a normal case and (b) an acute rejection case where the correlations experience variations indicative of renal rejection.

FIG. 4 illustrates application of PCA to the correlations. Panel (a) depicts the original Spearman correlation matrix and panel (b) illustrates the matrix after retaining significant correlations and discarding insignificant correlations.

### DETAILED DESCRIPTION OF THE PREFERRED EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter. As used herein, the term “about,” when referring to a value or to an amount is meant to encompass variations of +10% of the most precise digit in the value or amount (e.g., “about 1” refers to 0.9 to 1.1, “about 1.1” refers to 1.09 to 1.11, etc.). The term “substantially,” when modifying a term associated with a number, has the same meaning as “about” (e.g., “substantially perpendicular” to an element means an orientation with ±10% of 90 degrees with respect to that element).

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

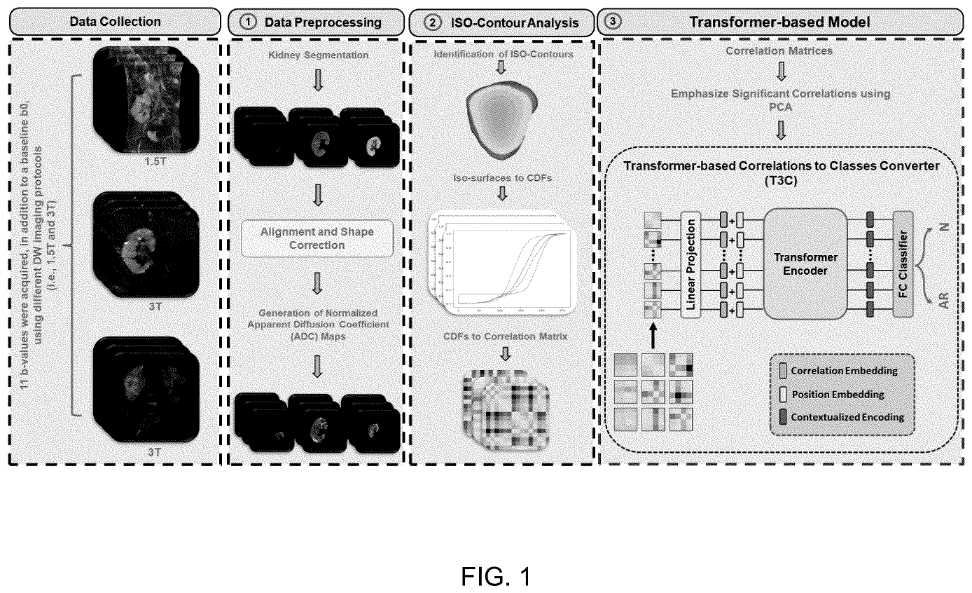

A transformer-based framework is introduced to predict acute renal transplant rejection using diverse DW-MRI scanning protocols (see FIG. 1). The frame-work begins with preprocessing, which yields segmented ADC maps. Then, iso-surfaces are generated to represent distinct kidney regions. Afterwards, the T3C model leverages the correlations of CDFs which correspond to these regions for a comprehensive prediction of kidney states.

### Preprocessing

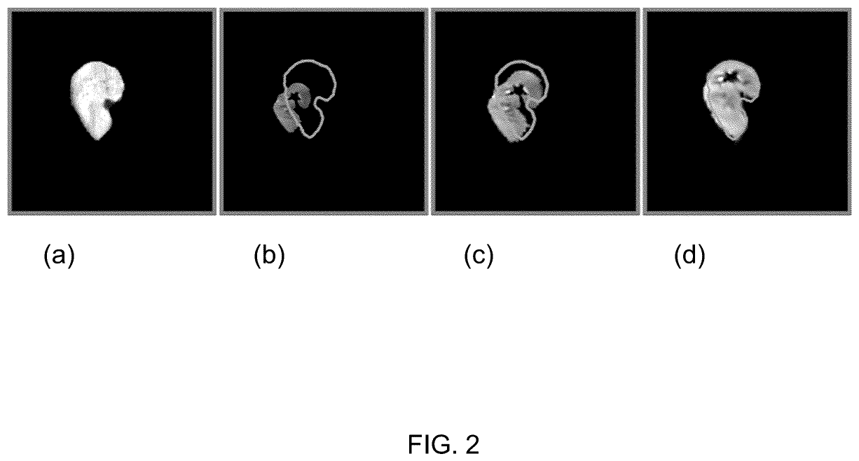

As depicted in FIG. 1, this step involves segmenting a plurality of medical images of a subject transplanted kidney to extract the kidney from each image. The plurality of medical images are DW-MRI scans of a subject kidney. A common challenge in DW-MRIs is the shape deformation that can lead to the alteration of MRI diffusion signals. The geometric shapes of anatomical structures can also affect the prediction accuracy. Therefore, the segmented kidneys undergo processing to align them and eliminate their deformation in intra-patient scenarios, such as due to breathing and/or heart beating, and to account for the kidney's variability due to inter-patient anatomical differences. Here, one of the 3D images is chosen as a reference image and all other 3D images are aligned to it in terms of scale, rotation, translation and shear by applying a 12-degree-of-freedom affine-based registration to correct for broad, inter-scan differences. To correct finer deformations, a non-rigid 3D B-spline transformation is then applied to address more subtle shape variations within the kidney due to breathing or anatomical differences (see FIG. 2). Afterwards, the registered kidneys are transformed to yield ADC maps using known techniques, providing a standardized functional representation. These ADC maps are then used as inputs for the next step.

### Iso-Surface Analysis

The disclosed non-invasive system and method for identifying potential abnormalities indicative of renal rejection utilizes correlations of CDFs which correspond to iso-surfaces of the kidney. Since the correlation measures the strength and direction of a linear relationship between two variables (in this case, the CDFs of iso-surfaces), the system and method focuses on the relationship patterns between distributions of different kidney regions using correlation rather than analyzing the raw imaging data directly. This approach mitigates the impact of scanner-induced variations on the raw imaging data values, which can be influenced by the specific characteristics of the scanner. A fast marching algorithm is employed to identify iso-surfaces although, in other embodiments, Graph-Cut-Based Surface Extraction or other methods may be used for iso-surface identification. The Spearman correlation is used for calculating correlations although, in other embodiments, Pearson Correlation, Kendall's Tau, or other methods may be used for determining correlations. The rationale behind using the Spearman correlation algorithm is its ability to assess monotonic relationships, determining whether there is a linear pattern or not between iso-surfaces.

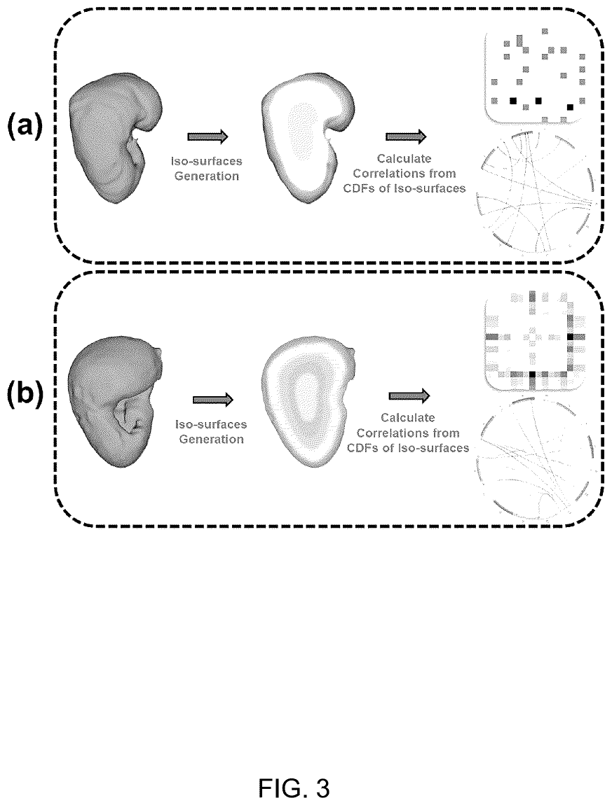

Upon visual inspection of FIG. 3, which displays the correlations 3D iso-surfaces for two patients (a) and (b), it is evident that the acutely rejected kidney, as shown in (b), exhibits variations in correlation when compared to the normal kidney, as illustrated in (a). In a normal kidney, the diffusion of water molecules tends to be more uniform across various segments, resulting in consistent ADC values throughout the tissue. This consistency leads to lower variation in correlation across the iso-surfaces, as the diffusion patterns remain relatively stable in different regions of the kidney. In contrast, a rejected kidney often exhibits heterogeneous diffusion patterns due to structural and functional changes associated with rejection, such as inflammation, fibrosis, or ischemia. These pathological changes create high diffusive movement in some segments, such as where tissue integrity is compromised, and lower diffusive movement in other segments, such as where inflammation or fibrosis restricts diffusion, resulting in greater variation in correlation across the iso-surfaces. This variation in diffusion is thus a marker for detecting potential rejection, as it signifies that the tissue no longer supports uniform water diffusion due to localized damage or abnormalities.

### Transformer-Based Correlations to Classes Converter (T3C)

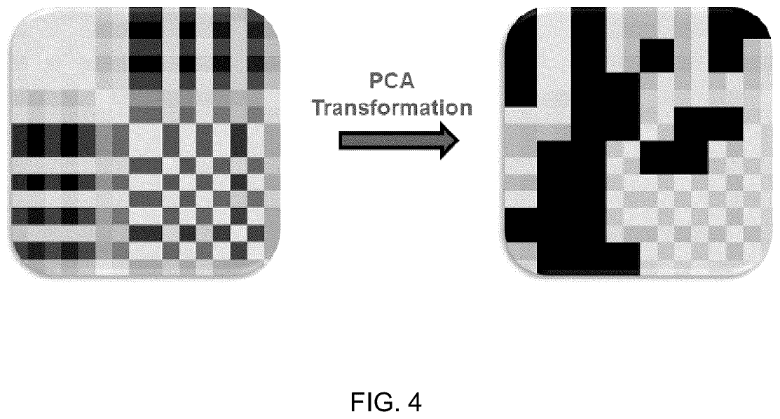

In the final stage, the correlations derived from the previous step undergo transformation via Principal Component Analysis (PCA). This is done to reduce the dimensionality of the obtained correlations, discarding those of lesser significance while preserving the essential ones (see FIGS. 1 and 4). This procedure aids in the development of a robust model that can accurately predict the status of transplanted kidneys, differentiating between normal function and instances of acute rejection. To achieve this, a T3C model was designed that learns to map correlations to class scores. Specifically, a correlation tensor x∈ [symbol] is defined, where N represents the number of b-values for each patient, R is the number of rows, and C is the number of columns. This tensor is reshaped and linearly transformed to produce a sequence of correlation embeddings x=[e₁ . . . , e_{N}]∈ [symbol] , where e∈ [symbol] . To maintain positional in-formation, learnable position embeddings p=[p₁ . . . , p_{N}]∈ [symbol] are added to the correlation embeddings, resulting in the input sequence of tokens z=x+p. The T3C model's transformer encoder, composed of H layers, processes the input sequences z, generating contextualized encodings z_{H}ϵ [symbol] . Each transformer layer includes a multi-headed self-attention (MSA) block followed by a point-wise Multi-Layer Perceptron (MLP) block, with layer normalization (LN) applied before and residual connections added after each block:

$${a}_{i - 1} = MSA ( LN ( {z}_{i - 1} ) ) + {z}_{i - 1} ,$$

$${z}_{i} = MLP ( LN ( {a}_{i - 1} ) ) + {a}_{i - 1} ,$$

where iϵ{1, . . . , H}. The self-attention mechanism computes queries Q∈ [symbol] , keys K∈ [symbol] , and values V∈ [symbol] , via three point-wise linear layers and then computes self-attention as:

$$MSA ( Q , K , V ) = soft max ( \frac{{QK}^{T}}{\sqrt{d}} ) V$$

The transformer encoder maps the input sequences to a contextualized encoding sequence z_{H}=[z_{H,1}, . . . , z_{H,N}] that contains rich salient information. Subsequently, zH is utilized by a Fully Connected (FC) classifier to obtain class scores corresponding to normal and acute rejection, and thus classifies the subject kidney as one of normal or acute rejection.

The classification process includes a training phase where the transformer model, including the FC classifier, learns to associate specific score patterns with normal and acute rejection cases. A typical training phase includes: (1) training the model on a dataset of DW-MRI images of kidneys captured at a plurality of different magnetic field strengths and labeled by medical professionals as either normal or acutely rejected, each training instance including the ADC-derived correlation data and the known classification label (normal or acutely rejected), (2) the transformer encoder and FC classifier learn to map input features (i.e., the correlation patterns across iso-surfaces) to specific class scores and, through backpropagation, the model adjusts its weights to minimize the difference between its predications and the true labels and develop an internal representation that assigns higher scores to patterns recognized by the model as either normal or rejected, (3) the output scores from the FC classifier represent probabilities indicating the likelihood of each class (normal or acutely rejected) and, through training, the model learns a decision boundary based on these scores to classify new instances, (4) after training, the model is tested on unseen data to validate that it can generalize and correctly classify kidneys based on the learned score patterns and confirm that the learned scores are predictive of normal and rejected states.

### Experiments and Results

The dataset used in this invention includes DW-MRI scans from 94 patients. Of these, 34 were obtained using a 1.5T SIGNA Horizon scanner, adhering to specific parameters for coronal DW-MR images. The scans included 11 b-values sequences and baseline b0 scans, capturing blood perfusion and water diffusion effects. Each sequence used a single DW-MRI direction with equal gradient amplitudes. Furthermore, 60 DW-MRI scans were performed using a 3T scanner, following similar protocols with minor adjustments due to different scanner models. Each scan also included 11 b-values and the baseline b0.

In one embodiment, an Adam optimizer with a learning rate of 0.001 over 100 epochs is used during the training phase of the transformer model and FC classifier to help adjust weights. Leave-one-out cross-validation is employed for evaluating the models, and the reported results represent the mean of ten runs. Additionally, L2 loss is utilized for training the model. The implementation was carried out using Py-Torch on a single NVIDIA Quadro P4000 GPU with 8 GB of memory.

Two main experiments were conducted: the first used classical ML classifiers to categorize correlations, while the second utilized the transformer-based model disclosed herein. As shown in Table 1, among the ML classifiers, Random Forest (RF) achieved the highest mean Accuracy (ACC) of 51.915%, mean Specificity (SPE) of 70.370%, and mean Area Under the Curve (AUC) of 48.685%. Decision Tree (DT) attained the highest mean Sensitivity (SEN) of 42.250% compared to Multi-Layer Perceptron (MLP) and Gradient Boosting (GB). However, the disclosed T3C model outperformed all others, boasting a mean ACC of 98.723%, mean SEN of 97%, mean SPE of 100%, and mean AUC of 98.5%, surpassing RF, DT, MLP, and GB. Furthermore, a Mann-Whitney statistical test revealed significant statistical superiority of the T3C model with a p-value less than 0.001 compared to other classifiers. These findings have profound implications for clinical practice, as accurate prediction of normalcy or acute rejection in transplanted kidneys is crucial for effective treatment planning and monitoring.

TABLE 1
A comparison of the classification results between the T3C model and
classical ML classifiers. The mean ± standard deviation, expressed
in percentage, along with the statistical significance, are reported.

| Classifier | ACC | SEN | SPE | AUC | Mann-Whitney |
|---|---|---|---|---|---|
| RF | 51.915 ± 3.184 | 27.000 ± 7.399 | 70.370 ± 5.617 | 48.685 ± 3.340 | 1.62 × 10⁻⁴ |
| DT | 41.596 ± 3.478 | 42.250 ± 4.931 | 41.111 ± 4.040 | 41.681 ± 3.538 | 1.62 × 10⁻⁴ |
| MLP | 44.787 ± 1.809 | 32.250 ± 2.839 | 54.074 ± 2.845 | 43.162 ± 1.777 | 1.46 × 10⁻⁴ |
| GB | 47.553 ± 1.069 | 28.750 ± 1.250 | 61.481 ± 1.814 | 45.116 ± 0.991 | 1.33 × 10⁻⁴ |
| T3C | 98.723 ± 1.241 | 97.000 ± 2.915 | 100.000 ± 0 | 98.500 ± 1.458 | — |

In summary, this disclosure introduces a novel framework for predicting both normal kidney function and instances of acute rejection. The framework leverages diverse DW-MRI datasets obtained through various scanning protocols, specifically, 1.5T and 3T. This approach effectively addresses the primary challenge of creating a system robust to variations in DW-MRI protocol across different scanners. The framework's remarkable sensitivity and specificity results establish its potential as a significant tool for treatment planning and monitoring patient responses to kidney transplants.

The disclosed computer-aided system and method system may be embodied in computer program instructions stored on a non-transitory computer readable storage medium configured to be executed by a computing system. The computing system utilized in conjunction with the computer-aided system described herein will typically include a processor in communication with a memory, and a network interface. Power, ground, clock, and other signals and circuitry are not discussed, but will be generally understood and easily implemented by those ordinarily skilled in the art. The processor, in some embodiments, is at least one microcontroller or general purpose microprocessor that reads its program from memory. The memory, in some embodiments, includes one or more types such as solid-state memory, magnetic memory, optical memory, or other computer-readable, non-transient storage media. In certain embodiments, the memory includes instructions that, when executed by the processor, cause the computing system to perform a certain action. Computing system also preferably includes a network interface connecting the computing system to a data network for electronic communication of data between the computing system and other devices attached to the network. In certain embodiments, the processor includes one or more processors and the memory includes one or more memories. In some embodiments, computing system is defined by one or more physical computing devices as described above. In other embodiments, the computing system may be defined by a virtual system hosted on one or more physical computing devices as described above.

The foregoing detailed description is given primarily for clearness of understanding and no unnecessary limitations are to be understood therefrom for modifications can be made by those skilled in the art upon reading this disclosure and may be made without departing from the spirit of the invention.

1. A computer-aided method for diagnosis of renal rejection comprises:
receiving a plurality of medical images of a subject transplanted kidney;
transforming each of the plurality of medical images into a corresponding apparent diffusion coefficient (ADC) map;
identifying, for each ADC map, iso-surfaces in the ADC map;
identifying correlations between iso-surfaces;
classifying, using a machine-learning classifier, the subject kidney as normal or acutely rejected based at least in part on the correlations.

2. The computer-aided method of claim 1, wherein the plurality of medical images of the subject transplanted kidney are a plurality of DW-MRI scans of the subject kidney.

3. The computer-aided method of claim 2, wherein the machine-learning classifier is trained on a data set of DW-MRI scans of normal kidneys and acutely rejected kidneys obtained at a plurality of different magnetic field strengths.

4. The computer-aided method of claim 1, further comprising, prior to said transforming, selecting one of the plurality of medical images to be a reference image and aligning the remaining images in the plurality of medical images to the reference image.

5. The computer-aided method of claim 1, wherein identifying correlations between iso-surfaces comprises identifying correlations between cumulative distribution functions of each iso-surface.

6. The computer-aided method of claim 1, further comprising, determining the significance of each correlation and discarding correlations of lesser significance prior to the machine-learning classifier receiving the correlations as input.

7. The computer-aided method of claim 1, further comprising, subsequent to identifying correlations between iso-surfaces and prior to said classifying, defining an input series of tokens, each token comprising learnable position embeddings and correlation embeddings derived from the identified correlations between iso-surfaces.

8. The computer-aided method of claim 7, wherein the machine learning classifier includes a transformer encoder and a classifier.

9. The computer-aided method of claim 8, wherein the encoder maps the input series of tokens to a contextualized encoding sequence, and wherein the classifier classifies the subject kidney as normal or acutely rejected based on the contextualized encoding sequence.

10. The computer-aided method of claim 7, wherein the transformer encoder includes a plurality of layers, each later including a multi-headed self-attention (MSA) block followed by a point-wise Multi-Layer Perceptron (MLP) block, with layer normalization (LN) applied before and residual connections added after each block.

11. A computer-aided system for diagnosis of renal rejection, the system comprising:
at least one non-transitory computer readable storage medium having computer program instructions stored thereon; and
at least one processor configured to execute the computer program instructions causing the processor to perform the following operations:
receiving a plurality of medical images of a subject transplanted kidney;
transforming each of the plurality of medical images into a corresponding apparent diffusion coefficient (ADC) map;
identifying, for each ADC map, iso-surfaces in the ADC map;
identifying correlations between iso-surfaces;
classifying, using a machine-learning classifier, the subject kidney as normal or acutely rejected based at least in part on the correlations.

12. The computer-aided system of claim 11, wherein the plurality of medical images of the subject transplanted kidney are a plurality of DW-MRI scans of the subject transplanted kidney.

13. The computer-aided system of claim 12, wherein the machine-learning classifier is trained on a data set of DW-MRI scans of normal kidneys and acutely rejected kidneys obtained at a plurality of different magnetic field strengths.

14. The computer-aided system of claim 11, further comprising, prior to said transforming, selecting one of the plurality of medical images to be a reference image and aligning the remaining images in the plurality of medical images to the reference image.

15. The computer-aided system of claim 11, wherein identifying correlations between iso-surfaces comprises identifying correlations between cumulative distribution functions of each iso-surface.

16. The computer-aided system of claim 11, further comprising, determining the significance of each correlation and discarding correlations of lesser significance prior to the machine-learning classifier receiving the correlations as input.

17. The computer-aided system of claim 11, further comprising, subsequent to identifying correlations between iso-surfaces and prior to said classifying, defining an input series of tokens, each token comprising learnable position embeddings and correlation embeddings derived from the identified correlations between iso-surfaces.

18. The computer-aided system of claim 17, wherein the machine learning classifier includes a transformer encoder and a classifier.

19. The computer-aided system of claim 18, wherein the encoder maps the input series of tokens to a contextualized encoding sequence, and wherein the classifier classifies the subject kidney as normal or acutely rejected based on the contextualized encoding sequence.

20. The computer-aided system of claim 17, wherein the transformer encoder includes a plurality of layers, each later including a multi-headed self-attention (MSA) block followed by a point-wise Multi-Layer Perceptron (MLP) block, with layer normalization (LN) applied before and residual connections added after each block
